(19) United States
(12) Patent Application Publication
(10) Pub. No.: US 2021/0052785 A1
(43) Pub. Date: Feb. 25, 2021

(54) Ear and Nose Secretion Extractor Apparatus

(71) Applicant: Iven Ernest Milton, JR., New Rochelle, NY (US)

(72) Inventor: Iven Ernest Milton, JR., New Rochelle, NY (US)

(21) Appl. No.: 16/546,892

(22) Filed: Aug. 21, 2019

(51) Int. Cl. A61M 1/00

(52) U.S. Cl. CPC A61M 1/0003; A61M 2205/8206; A61M 2205/8237; A61M 2205/587; A61M 2210/0662; A61M 2209/088; A61M 2210/0618

(57) ABSTRACT

An ear and nose secretion extractor apparatus for removing mucus and cerumen includes a headband configured to be worn on a crown of a user's head. A housing is coupled to the headband and defines a housing cavity. A pump and a battery are coupled to the housing within the housing cavity. A nasal arm is coupled to the housing. A pair of nasal collection chambers is coupled to the nasal arm. A pair of nasal nozzles is coupled to the pair of nasal collection chambers. A pair of aural arms is coupled to the headband. A pair of aural collection chambers is coupled to the pair of aural arms. The pair of aural collection chambers is in fluid communication with the pump. The pump creates suction through the nasal nozzles. The pair of aural nozzles is configured to be inserted into a user's ears.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR Not Applicable

### BACKGROUND OF THE INVENTION

### (1) Field of the Invention

### (2) Description of Related Art Including Information Disclosed Under 37 CFR 1.97 and 1.98

The disclosure and prior art relates to health tools and more particularly pertains to a new health tool for removing mucus and cerumen.

### BRIEF SUMMARY OF THE INVENTION

An embodiment of the disclosure meets the needs presented above by generally comprising a headband comprising a front portion, a back portion, a left portion, and a right portion. The headband is configured to be worn on a crown of a user's head. A housing is coupled to the front portion of the headband and defines a housing cavity. A pump and a battery are coupled to the housing within the housing cavity. The battery is in operational communication with the pump. A charge port is coupled to the housing and is in operational communication with the battery. A power button is coupled to the housing and is in operational communication with the pump and the battery. A nasal arm is coupled to the housing. A nasal base is coupled to the nasal arm. A pair of nasal collection chambers is coupled to the nasal base and is in fluid communication with the pump. A pair of nasal nozzles is coupled to the pair of nasal collection chambers. The pair of nasal nozzles is configured to be inserted into a user's nostrils. A pair of aural arms is coupled to the headband. The pair of aural arms comprises a left aural arm and a right aural arm coupled to the left portion and the right portion of the headband. A pair of aural bases is coupled to the pair of aural arms. A pair of aural collection chambers is coupled to the pair of aural bases. The pair of aural collection chambers is in fluid communication with the pump. A pair of aural nozzles is coupled to the pair of aural collection chambers. The pair of aural nozzles is configured to be inserted into a user's ears.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an ear and nose secretion extractor apparatus according to an embodiment of the disclosure.

FIG. 2 is a front elevation view of an embodiment of the disclosure.

FIG. 3 is an exploded view of an embodiment of the disclosure.

FIG. 4 is an in-use view of an embodiment of the disclosure.

FIG. 5 is a block diagram view of an embodiment of the disclosure.

### DETAILED DESCRIPTION OF THE INVENTION

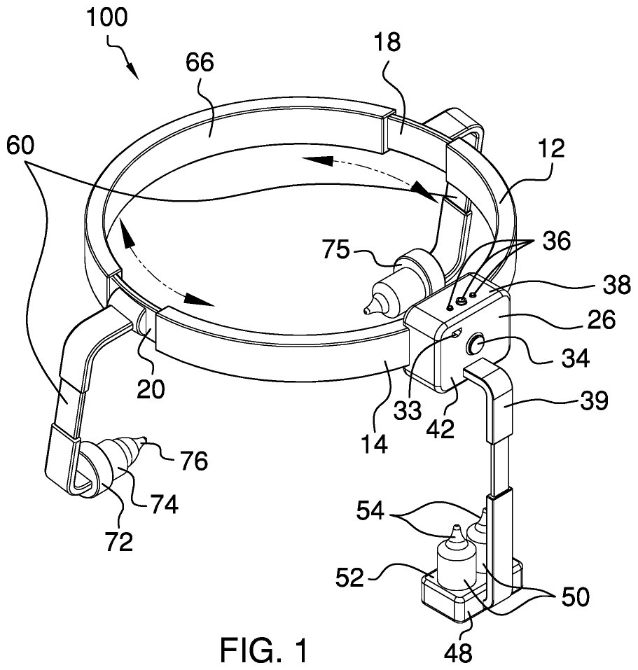

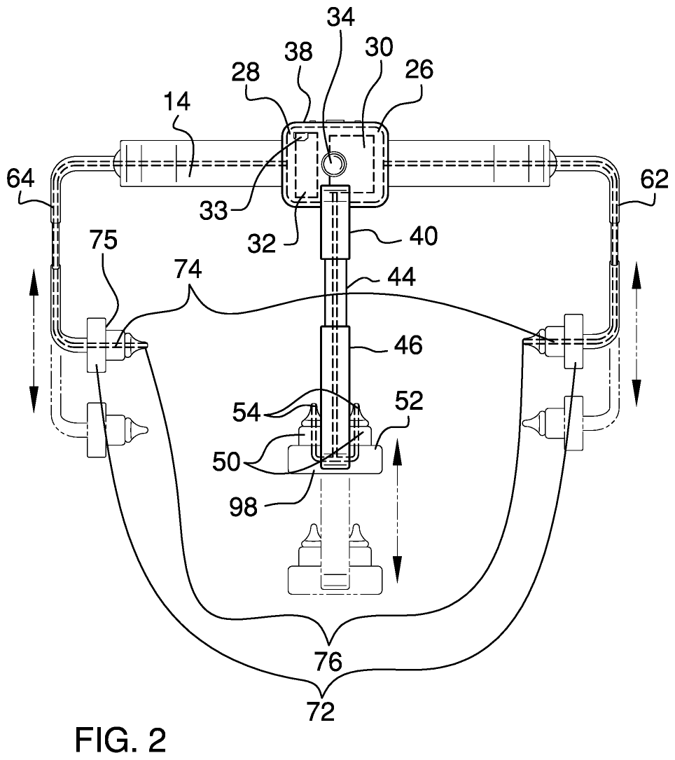

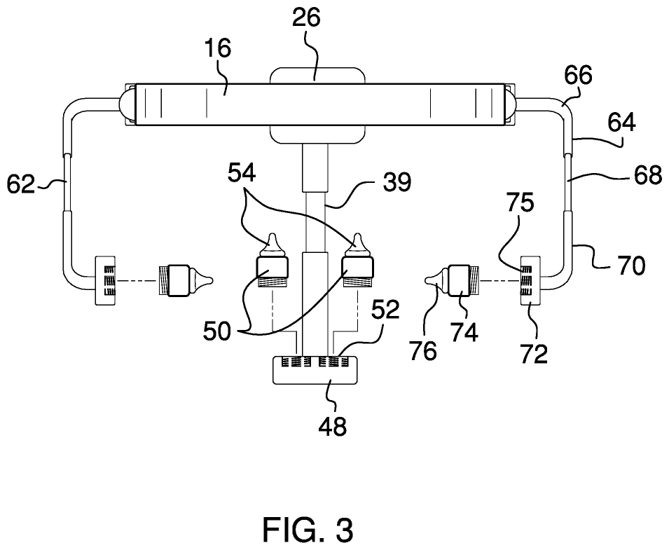

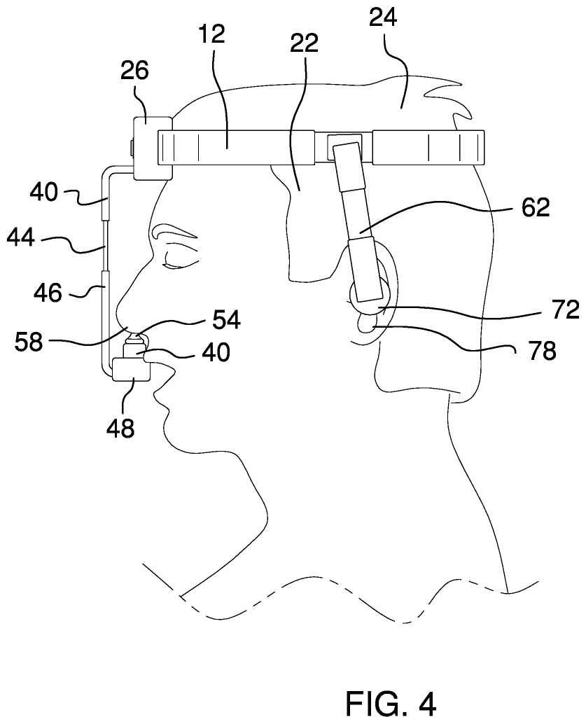

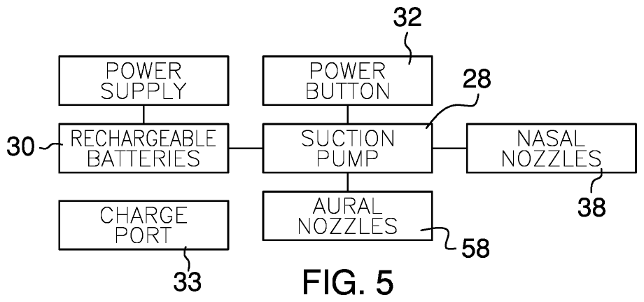

With reference now to the drawings, and in particular to FIGS. 1 through 5 thereof, a new health tool embodying the principles and concepts of an embodiment of the disclosure and generally designated by the reference numeral 10 will be described.

As best illustrated in FIGS. 1 through 5, the ear and nose secretion extractor apparatus 10 generally comprises a headband 12 comprising a front portion 14, a back portion 16, a left portion 18, and a right portion 20. The headband 12 is adjustable with each of the left portion 18 and the right portion 20 being slidably engaged within the front portion 14 and the back portion 16. The headband 12 is configured to be worn on a crown 22 of a user's head 24. A housing 26 is coupled to the front portion of the headband 12 and defines a housing cavity 28. A pump 30 is coupled within the housing cavity 28. A battery 32 is coupled within the housing cavity 28. The battery 32 is in operational communication with the pump 30. A charge port 33 is coupled to the housing 26 and is in operational communication with the battery 32. A power button 34 is coupled to the housing 26 and is in operational communication with the pump 30 and the battery 32. A plurality of status lights 36 is coupled to the housing 26. The plurality of status lights 36 is coupled to a housing top side 38 of the housing 26. The plurality of status lights 36 is in operational communication with the pump 30 and the battery 32. A nasal arm 39 is coupled to the housing 26. The nasal arm 39 comprises an L-shaped nasal proximal portion 40 coupled to a housing front side 42 of the housing 26 below the power button 34, a straight nasal medial portion 44 coupled to the nasal proximal portion 40, and an L-shaped nasal distal portion 46 slidably coupled to the nasal medial portion 44. A nasal base 48 is coupled to the nasal arm 39. The nasal base 48 is rectangular prismatic. A pair of nasal collection chambers 50 is coupled to the nasal base 48. Each of the nasal collection chambers 50 is cylindrical and threadably engageable within a nasal top side 52 of the nasal base 48. The pair of nasal collection chambers 50 is in fluid communication with the pump 30. A pair of nasal nozzles 54 is coupled to the pair of nasal collection chambers 50. The pair of nasal nozzles 54 is configured to be inserted into a user's nostrils 58.

A pair of aural arms 60 is coupled to the headband. The pair of aural arms 60 comprises a left aural arm 62 and a right aural arm 64 coupled to the left portion 18 and the right portion 20 of the headband 12, respectively. Each aural arm 60 comprises an L-shaped aural proximal portion 66 coupled to the headband 12, a straight aural medial portion 68 coupled to the aural proximal portion 66, and an L-shaped aural distal portion 70 slidably coupled to the aural medial portion 68. A width of the aural distal portion 70 is less than a width of the aural proximal portion 66. A pair of aural bases 72 is coupled to the pair of aural arms 60. The pair of aural bases 72 is coupled to the aural distal portion 70 of the pair of aural arms 60. Each of the aural bases 72 is cylindrical. A pair of aural collection chambers 74 is coupled to the pair of aural bases 72. Each of the aural collection chambers 74 is cylindrical and threadably engageable with an aural inner face 75 of each of the aural bases 72. Each aural collection chamber 74 has a diameter less than a diameter of each of the aural bases 72. The pair of aural collection chambers 74 is in fluid communication with the pump 30. A pair of aural nozzles 76 is coupled to the pair of aural collection chambers 74. The pair of aural nozzles 76 is configured to be inserted into a user's ears 78.

In use, the headband 12 is placed on the crown 22 of the user's head 25. The aural nozzles 76 are inserted into the user's ears 78 and the nasal nozzles 54 are inserted into a user's nostrils 58. The power button 34 is depressed and the pump 30 sucks nasal and aural residue into the pair of nasal collection chambers 50 and the pair of aural collection chambers 74, respectively. The aural nozzles 76 and the nasal nozzles 54 may be removed to clean out the pair of nasal collection chambers 50 and the pair of aural collection chambers 74.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ear and nose secretion extractor apparatus comprising:
a headband, the headband comprising a front portion, a back portion, a left portion, and a right portion, the headband being configured to be worn on a crown of a user's head;
a housing coupled to the headband, the housing being coupled to the front portion, the housing defining a housing cavity;
a pump coupled to the housing, the pump being coupled within the housing cavity;
a battery coupled to the housing, the battery being coupled within the housing cavity, the battery being in operational communication with the pump;
a charge port coupled to the housing, the charge port being in operational communication with the battery;
a power button coupled to the housing, the power button being in operational communication with the pump and the battery;
a nasal arm coupled to the housing;
a nasal base coupled to the nasal arm;
a pair of nasal collection chambers coupled to the nasal base, the pair of nasal collection chambers being in fluid communication with the pump;
a pair of nasal nozzles coupled to the pair of nasal collection chambers, the pair of nasal nozzles being configured to be inserted into a user's nostrils;
a pair of aural arms coupled to the headband, the pair of aural arms comprising a left aural arm and a right aural arm coupled to the left portion and the right portion of the headband, respectively;
a pair of aural bases coupled to the pair of aural arms;
a pair of aural collection chambers coupled to the pair of aural bases, the pair of aural collection chambers being in fluid communication with the pump; and
a pair of aural nozzles coupled to the pair of aural collection chambers, the pair of aural nozzles being configured to be inserted into a user's ears.

2. The ear and nose secretion extractor apparatus of claim 1 further comprising the headband being adjustable with each of the left portion and the right portion being slidably engaged within the front portion and the back portion.

3. The ear and nose secretion extractor apparatus of claim 1 further comprising the nasal arm comprising an L-shaped nasal proximal portion coupled to the housing, a straight nasal medial portion coupled to the nasal proximal portion, and an L-shaped nasal distal portion slidably coupled to the nasal medial portion, each aural arm comprising an L-shaped aural proximal portion coupled to the headband, a straight aural medial portion coupled to the aural proximal portion, and an L-shaped aural distal portion slidably coupled to the aural medial portion.

4. The ear and nose secretion extractor apparatus of claim 3 further comprising a width of the aural distal portion being less than a width of the aural proximal portion.

5. The ear and nose secretion extractor apparatus of claim 1 further comprising the nasal base being rectangular prismatic, each of the nasal collection chambers being cylindrical and threadably engageable within a nasal top side of the nasal base.

6. The ear and nose secretion extractor apparatus of claim 1 further comprising each of the aural bases being cylindrical, each of the aural collection chambers being cylindrical and threadably engageable with an aural inner face of each of the aural bases, each aural collection chamber having a diameter less than a diameter of each of the aural bases.

7. The ear and nose secretion extractor apparatus of claim 1 further comprising the nasal arm being coupled to a housing front side of the housing, the power button being coupled above the nasal arm.

8. The ear and nose secretion extractor apparatus of claim 1 further comprising a plurality of status lights coupled to a housing top side of the housing, the plurality of status lights being in operational communication with the pump and the battery.

9. An ear and nose secretion extractor apparatus comprising:
a headband, the headband comprising a front portion, a back portion, a left portion, and a right portion, the headband being adjustable with each of the left portion and the right portion being slidably engaged within the front portion and the back portion, the headband being configured to be worn on a crown of a user's head;
a housing coupled to the headband, the housing being coupled to the front portion, the housing defining a housing cavity;
a pump coupled to the housing, the pump being coupled within the housing cavity;
a battery coupled to the housing, the battery being coupled within the housing cavity, the battery being in operational communication with the pump;
a charge port coupled to the housing, the charge port being in operational communication with the battery;
a power button coupled to the housing, the power button being in operational communication with the pump and the battery;
a plurality of status lights coupled to the housing, the plurality of status lights coupled to a housing top side of the housing, the plurality of status lights being in operational communication with the pump and the battery;
a nasal arm coupled to the housing, the nasal arm comprising an L-shaped nasal proximal portion coupled to a housing front side of the housing below the power button, a straight nasal medial portion coupled to the nasal proximal portion, and an L-shaped nasal distal portion slidably coupled to the nasal medial portion;
a nasal base coupled to the nasal arm, the nasal base being rectangular prismatic;
a pair of nasal collection chambers coupled to the nasal base, each of the nasal collection chambers being cylindrical and threadably engageable within a nasal top side of the nasal base, the pair of nasal collection chambers being in fluid communication with the pump;
a pair of nasal nozzles coupled to the pair of nasal collection chambers, the pair of nasal nozzles being configured to be inserted into a user's nostrils;
a pair of aural arms coupled to the headband, the pair of aural arms comprising a left aural arm and a right aural arm coupled to the left portion and the right portion of the headband, respectively, each aural arm comprising an L-shaped aural proximal portion coupled to the headband, a straight aural medial portion coupled to the aural proximal portion, and an L-shaped aural distal portion slidably coupled to the aural medial portion, a width of the aural distal portion being less than a width of the aural proximal portion;
a pair of aural bases coupled to the pair of aural arms, the pair of aural bases being coupled to the aural distal portion of the pair of aural arms, each of the aural bases being cylindrical;
a pair of aural collection chambers coupled to the pair of aural bases, each of the aural collection chambers being cylindrical and threadably engageable with an aural inner face of each of the aural bases, each aural collection chamber having a diameter less than a diameter of each of the aural bases, the pair of aural collection chambers being in fluid communication with the pump; and
a pair of aural nozzles coupled to the pair of aural collection chambers, the pair of aural nozzles being configured to be inserted into a user's ears.
